(19) United States
(12) Patent Application Publication
(10) Pub. No.: US 2009/0306534 A1
(43) Pub. Date: Dec. 10, 2009

(54) SYSTEMS AND METHODS FOR PREDICTING EFFECTIVENESS IN THE TREATMENT OF PSYCHIATRIC DISORDERS, INCLUDING DEPRESSION

(75) Inventor: Diego A. Pizzagalli, Winchester, MA (US)

Correspondence Address: Harvard University & Medical School;c/o Wolf, Greenfield & Sacks, P.C., 600 Atlantic Avenue, Boston, MA, 02210-2206, US

(73) Assignee: President and Fellows of Harvard College, Cambridge, MA (US)

(21) Appl. No.: 12/225,943

(22) PCT Filed: Apr. 3, 2007
(86) PCT No.: PCT/US07/08107

§ 371 Date: Mar. 11, 2009

Related U.S. Application Data
(60) Provisional application No. 60/788,656, filed on Apr. 3, 2006.

(51) Int. Cl. A61B 5/0476; G09B 19/00

(52) U.S. Cl. 600/544; 434/236

(57) ABSTRACT

The present invention pertains to the evaluation and/or treatment of psychopathological diseases. In one aspect, information regarding brain activity, coupled with behavior tests (e.g., determining performance in a computer-based task), may be used to predict the response of a subject to psychological treatment, e.g., with a psychoactive drug. For example, the subject may be one suffering from depression, or other disturbances of the rostral anterior cingulate cortex. Another aspect of the present invention is directed to methods for analyzing neurobiological predictors through integration of information gathered from one or more levels of analyses: (1) behavior, (2) brain function, and/or (3) genes. In one aspect, the methods of the present invention can comprise any one of these components (i.e., behavior, brain function, and genes), or a combination of two or more of these components, and/or other components. Through these methods, development of novel algorithms for improving the ability to identify biological surrogate markers of treatment response are disclosed, according to certain embodiments of the invention. Still other aspects of the present invention are directed to systems and methods for implementing such evaluation techniques, analyzing such evaluation techniques, promotion of such evaluation techniques, and the like.

### FIELD OF INVENTION

The present invention pertains to the evaluation and/or treatment of psychopathological diseases.

### BACKGROUND

Major depressive disorder (MDD) is a common, recurrent, and disabling disorder. Epidemiological studies show that depressive disorders are the leading cause of years lived with disability, resulting in a global loss of 13 million years each year worldwide, 1.5 million of those in the U.S. alone, where the lifetime prevalence of depression is 17%. MDD remains difficult to treat, despite the wide range of antidepressants available. Studies show that up to 34% of patients fail to respond to antidepressant (AD) treatments, 46% fail to achieve full remission, and 50% will suffer from a recurrence. Moreover, a delay of 2-3 weeks is typically observed before the first effects of AD treatment emerge. A better understanding of treatment mechanisms and identification of reliable predictors of AD response would constitute major progress in the battle against MDD. Discovery of biological predictors at early stages of, or even before treatment, would allow clinicians to promptly identify patients who will likely fail to respond, and thus facilitate prompt implementation of other therapeutic strategies. In terms of patients' suffering and economic costs, the implications would be vast.

Despite the availability of many AD drugs, a substantial proportion of depressed patients fail to achieve a satisfactory response. In addition to nonresponders, the proportion of patients who drop out because of treatment-emergent side effects is substantial. Several factors may contribute to individual differences in drug response, including age, gender, medical comorbidity, or interaction with other drugs. Unfortunately, to date, attempts to identify clinical or sociodemographic features predicting AD treatment have met with very limited success. Regrettably, in clinical practice, treatment often follows a trial-and-error approach. An improved ability to predict in advance which patient will fail to respond to treatment would have a number of consequences, including a shorter time period in which depression is poorly controlled (decreasing the risk of suicide), an improved functioning and quality of life, and/or a reduction in healthcare costs.

### SUMMARY OF THE INVENTION

The present invention pertains to the evaluation and/or treatment of psychopathological diseases. The subject matter of the present invention involves, in some cases, interrelated products, alternative solutions to a particular problem, and/or a plurality of different uses of one or more systems and/or articles.

In one aspect, the present invention is directed toward a translational approach inspired by recent advances in affective neuroscience implicating the rostral anterior cingulate cortex (ACC) in treatment response. This may have the potential to shed new light on mechanisms leading to successful AD treatment.

Some methods of the present invention involve one or more of the following components: a behavioral & electrophysiological component, a neuroimaging component, and/or a genetic component.

One aspect of the present invention is directed to methods for analyzing neurobiological predictors through integration of information gathered from one or more levels of analyses: (1) behavior, (2) brain function, and/or (3) genes. In one aspect, the methods of the present invention can comprise any one of these components (i.e., behavior, brain function, and genes), or a combination of two or more of these components, and/or other components. Through these methods, development of novel algorithms for improving the ability to identify biological surrogate markers of treatment response can be effected, according to certain embodiments of the invention.

One aspect of the invention is directed to a method for predicting the success or failure of a given treatment for treating individuals diagnosed with or predisposed toward a psychiatric disorder. The method, in one set of embodiments, includes an act of analyzing behavior of an individual, where the behavior analysis is directed toward rostral ACC function of the individual. According to another set of embodiments, the method includes an act of analyzing brain function of an individual, wherein the function is analyzed using an electroencephalographic probe of rostral ACC function. In yet another set of embodiments, the method includes an act of analyzing brain function of an individual, where the brain function is analyzed using a hemodynamic probe of rostral ACC function. The method, according to still another set of embodiments, includes an act of analyzing a genome of an individual, where the genome analysis is directed toward 5-HT_{1A}, TPH-2, FKBP5, BDNF, and/or 5-HTTLPR genes.

In another set of embodiments, the method includes integration of information gathered from one or more levels of analyses, where the levels of analyses includes rostral ACC function, brain function using a hemodynamic probe, and/or genome analysis, wherein said genome analysis is directed toward 5-HT_{1A}, TPH-2, FKBP5, BDNF, and/or 5-HTTLPR genes.

The method, in another aspect, is directed to a method of diagnosing clinical depression in a subject. In one set of embodiments, the method includes acts of determining activity of at least a portion of the anterior cingulate cortex of the subject, determining an ability of the subject to respond to negative feedback and/or adjust behavior after committing errors, and diagnosing the subject as having clinical depression based on both the determination of the activity of the anterior cingulate cortex of the subject and the determination of the ability of the subject to respond to negative feedback and/or adjust behavior after committing errors.

In another set of embodiments, the method includes acts of determining activity of the anterior cingulate cortex of the subject, determining an ability of the subject to respond to negative feedback and/or adjust behavior after committing errors, entering the determinations into a computer, and receiving, from the computer, a probability assessment that the subject has clinical depression. The method, in still another set of embodiments, includes acts of determining activity of the anterior cingulate cortex of the subject, determining an ability of the subject to respond to negative feedback and/or adjust behavior after committing errors, entering the determinations into a computer, and receiving, from the computer, a probability assessment that the subject has clinical depression.

The method, in one aspect, includes acts of receiving a determination of activity of the anterior cingulate cortex of a subject, receiving a determination of the subject to respond to negative feedback and/or adjust behavior after committing errors, and combining the determinations into a combined score. In another aspect, the method includes acts of determining activity of a portion of the brain of a subject, determining an ability of the subject to respond to negative feedback and/or adjust behavior after committing errors, and diagnosing the subject as having clinical depression based on both the determination of the activity of the portion of the brain of the subject and the determination of the ability of the subject to respond to negative feedback and/or adjust behavior after committing errors. The method, in still another aspect, includes acts of determining activity of a portion the brain of a subject using tomography, determining an ability of the subject to respond to negative feedback and/or adjust behavior after committing errors, and diagnosing the subject as having clinical depression based on both the determination of the activity of the brain of the subject and the determination of the ability of the subject to respond to negative feedback and/or adjust behavior after committing errors.

Yet another aspect of the invention is directed to an article including a computer-readable medium having a program stored thereon. In one set of embodiments, the program may include instructions for, when executed, causing a computer-driven system to perform acts of receiving a determination of activity of the anterior cingulate cortex of a subject, receiving a determination of the subject to respond to negative feedback and/or adjust behavior after committing errors, combining the determinations into a combined score, and reporting the combined score. In another set of embodiments, the program may include instructions for, when executed, causing a computer-driven system to perform acts of determining activity of the anterior cingulate cortex of a subject, determining ability of the subject to respond to negative feedback and/or adjust behavior after committing errors, and identifying the subject as having clinical depression based on both the determination of the activity of the anterior cingulate cortex of the subject and the determination of the ability of the subject to respond to negative feedback and/or adjust behavior after committing errors.

Other advantages and novel features of the present invention will become apparent from the following detailed description of various non-limiting embodiments of the invention when considered in conjunction with the accompanying figures. For a better understanding of the present invention, together with other and further objects thereof, reference is made to the accompanying drawings and detailed description and its scope will be pointed out in the appended claims.

In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1 illustrates a model of the connections between different notions, according to one embodiment of the invention;

FIG. 2A-2C illustrate rostral anterior cingulate cortex activity, and a correlation between rostral anterior cingulate cortex activity and percentage Beck Depression Inventory (BDI) score change, in accordance with another embodiment of the invention;

FIG. 3 shows accuracy scores for low and high BDI subjects during positive or negative performance feedback, in one embodiment of the invention;

FIGS. 4A-4B illustrate accuracy scores and ERP waveforms (plotted at Cz, cingulate cortex) after incorrect and correct trials in MDD subjects, in another embodiment of the invention;

FIGS. 5A-5D illustrate fMRI studies of the anterior cingulate cortex, in another embodiment of the invention;

FIGS. 6A-6B illustrate mean accuracy for low and high BDI subjects, in one embodiment of the invention;

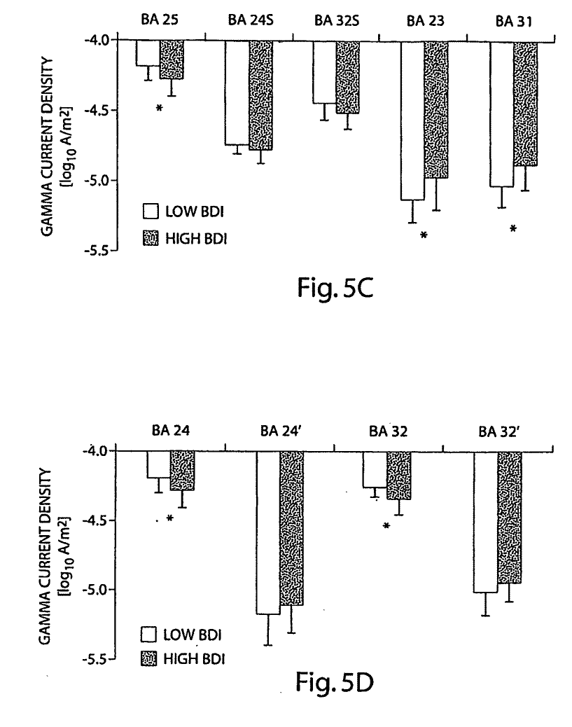

FIG. 7 illustrates a correlation between post-error adjustment effects and gamma current densities, in another embodiment of the invention;

FIG. 8 illustrates a power analysis according to one embodiment of the invention; and

FIG. 9 illustrates rostral ACC regions implicated in various tasks, in yet another embodiment of the invention.

### DETAILED DESCRIPTION

The present invention pertains to the evaluation and/or treatment of psychopathological diseases. In one aspect, information regarding brain activity, coupled with behavior tests (e.g., determining performance in a computer-based task), may be used to predict the response of a subject to psychological treatment, e.g., with a psychoactive drug. For example, the subject may be one suffering from depression, or other disturbances of the rostral anterior cingulate cortex. Another aspect of the present invention is directed to methods for analyzing neurobiological predictors through integration of information gathered from one or more levels of analyses: (1) behavior, (2) brain function, and/or (3) genes. In one aspect, the methods of the present invention can comprise any one of these components (i.e., behavior, brain function, and genes), or a combination of two or more of these components, and/or other components. Through these methods, development of novel algorithms for improving the ability to identify biological surrogate markers of treatment response are disclosed, according to certain embodiments of the invention. Still other aspects of the present invention are directed to systems and methods for implementing such evaluation techniques, analyzing such evaluation techniques, promotion of such evaluation techniques, and the like.

Several aspects of the present invention are directed toward methods that incorporate recent conceptual and methodological advances in affective neuroscience and molecular genetics in the study of pressing clinical issues. A conceptual framework is also presented, in various embodiments, for appropriately developing, assessing, interpreting, and/or reframing hypotheses about neurobiological predictors of treatment response. Also disclosed herein are paradigms, approaches, and proof-of-principle studies directed toward the goal of improving a practitioner's ability to predict treatment response in MDD, according to certain embodiments of the present invention. For example, the use of laboratory-based and biological approaches to probe a brain region, the rostral ACC, may reveal novel information about functional mechanisms for fostering efficacious treatment response.

One set of embodiments of the present invention is directed to methods for analyzing neurobiological predictors through integration of information gathered from one or more levels of analyses of the following components: (1) behavior, (2) brain function, and/or (3) genes, optionally with other components. In one embodiment, methods of the present invention can comprise any one of these components, or a combination of two or more of these components and/or other components. For example, an analysis may include behavioral and brain function components, behavioral and genetic components, and/or brain function and behavioral components. Through these methods, development of novel algorithms for improving the ability to identify biological surrogate markers of treatment response can be effected in certain embodiments of the invention. In one embodiment, the methods of the present invention can be implemented by a system including one or more processors and one or more computer usable media having computer readable code embodied therein. The computer readable code embodied in one or more computer usable media can cause the one or more processors to execute various methods of the present invention.

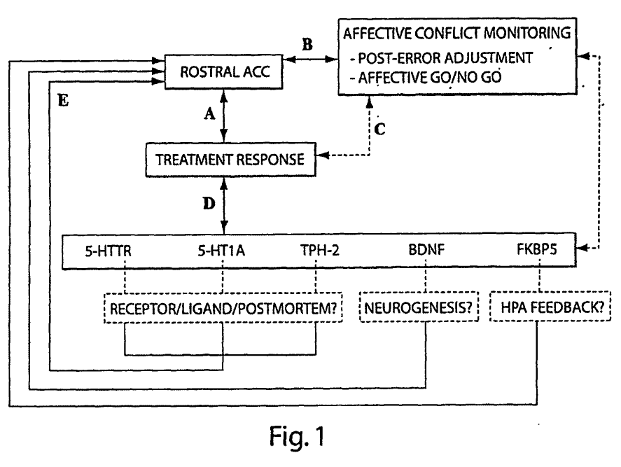

Summarized in FIG. 1 are some of the notions that support certain embodiments of the present invention. These notions include: (i) treatment response is associated with increased pre-treatment rostral ACC activity; (ii) the rostral ACC plays an important role in affective conflict monitoring; (iii) affective conflict monitoring may mediate treatment response; (iv) genetic factors can be important mediators of antidepressant response; and/or (v) the effects of genetic factors implicated in treatment response may be expressed in prefrontal and cingulate regions implicated in the pathophysiology and treatment of depression. Thus, in one set of embodiments, the present invention includes behavioral, electrophysiological, hemodynamic, and/or genetic approaches. For example, in one embodiment, the present invention may be used to determine neurobiological substrates linked to lack of response to an antidepressant treatment, for instance, a standard, first-line antidepressant treatment such as a SSRI, e.g., escitalopram.

In another set of embodiments, such analyses may be determined in conjunction with treatments of depression, for example, antidepressants (e.g., a first-line or standard antidepressant), such as SSRIs (selective serotonin reuptake inhibitors), e.g., escitalopram. As resting activity in the ACC may predict individual differences in post-error behavioral adjustments, when a comparison is made with responders to antidepressant (AD) treatments, nonresponders may show lower post-error adaptation effects, higher error-related negativity (ERN), and/or lower error positivity (Pe).

Incorporated herein by reference is U.S. Provisional Patent Application Ser. No. 60/788,656, filed Apr. 3, 2006, entitled “Methods for Predicting Effectiveness in the Treatment of Psychiatric Disorders,” by Pizzagalli, et al.

Certain aspects of the invention are directed to the identification and/or diagnosis of major depressive disorder (MDD) or clinical depression in human subjects. Without wishing to be bound by any theory, it is believed that the anterior cingulate cortex, including the rostral anterior cingulate cortex, is involved with regulating emotion and mood, including depression such as MDD. Accordingly, in some aspects, by determining the state or condition of the anterior cingulate cortex, using direct and/or indirect methods, a subject can be diagnosed as having or being at risk for depression, including MDD or clinical depression. Furthermore, in certain embodiments of the invention, a subject's genetic make-up may also be considered, including certain single nucleotide polymorphisms (SNPs) that have been linked to depression. Thus, for example, the activity of the anterior cingulate cortex, the ability of the subject to respond to negative feedback and/or commission of an error, and/or the presence or absence of certain genes or SNPs can be used to identify or diagnose depression in the subject and/or predict the response to antidepressant treatment.

In one aspect of the invention, the activity of at least a portion of the brain of a subject is determined, for example, the anterior cingulate cortex (or a portion thereof, such as the rostral anterior cingulate cortex). As used herein, the term “determining” refers to both qualitative as well as quantitative measurements (e.g., the amount or degree of activity). In some cases, the amount of activity may be determined relative to a part of the brain, or to the brain as a whole in a resting state. In one set of embodiments, the local activity of a portion of the brain may be determined, for example, using tomographic techniques, as discussed below.

Examples of techniques that can be used to determine activity of the brain (e.g., neuronal activity) include, but are not limited to, electroencephalography (EEG) or event-related potential (ERP) measurements. An ERP is a measure of a brain response, typically electrical, that is the result of a thought or perception. EEG typically involves placing a number of electrodes on various parts of the brain to measure electrical activity, and includes several related techniques such as quantitative EEG (QEEG) or hemoencephalography (HEG). In some cases, using tomographic techniques or the like, the activity of a portion of the brain, such as the anterior cingulate cortex, may be determined, and in some cases, determined as a function of time. Non-limiting examples of EEG techniques able to at least partially resolve brain activity include low-resolution electromagnetic tomography (LORETA) or stereoelectroencephalography (SEEG). Those of ordinary skill in the art will be aware of different EEG or ERP techniques that can be used.

Other examples of techniques that can be used to determine activity of the brain include, but are not limited to, magnetoencephalography (MEG), magnetic resonance imaging such as functional magnetic resonance imaging (fMRI), positron emission tomography (PET) such as single photon positron emission tomography (SPECT), or the like. In some cases, more than one of the techniques described herein may be used to determine brain activity. Those of ordinary skill in the art are aware of these and other techniques that can be used to determine brain activity, and in some cases, brain activity in a portion of the brain, such as the anterior cingulate cortex. In some cases, tomographic techniques, which involve mathematical manipulation of data to determine spatial distributions, may be used to analyze data gathered from one or more of these techniques to determine activity of portions of the brain. As another example, electrophysiological (EEG/ERP) and/or behavioral probes for determining ACC function, such as rostral ACC function can be used. As mentioned, ACC function has been implicated in treatment response in major depression.

In another aspect of the present invention, the ability of the subject to respond to negative feedback and/or commission of an error is determined. For instance, in one embodiment, the ERN (error-related negativity) of a subject may be assessed in some fashion. Typically, this is determined by administering one or more tests to the subject, and evaluating the results. Without wishing to be bound by any theory, it is believed that the anterior cingulate cortex is involved with various rational cognitive functions, such as reward anticipation, decision-making, action monitoring, and emotion. Accordingly, by administering one or more tests that measure the ability of the subject to respond to negative feedback, the state or condition of the anterior cingulate cortex can be determined, at least in part.

An example of a test that determines the ability of the subject to respond to negative feedback and/or adjust behavior after committing errors is the Eriksen Flanker Task, which generally involves identifying whether a series of symbols are the same or different, for example, whether arrows are pointing in the same or different directions, whether a target letter within a string of letters were the same or different (e.g., “H” and “S” in strings such as HHHHH, SSSSS, SSHSS, HHSHH), etc. Other non-limiting examples include the Stroop Task (identifying words printed in one color but reciting a different color), the Counting Stroop Task (counting the number of times words identifying a number are shown, where the identified number may not be equal to the number of times it appears, e.g., “one one one”), the Emotional Counting Stroop (counting the number of times words having emotional content appear), the Directional Stroop Task (identifying words based on their physical location, which can be inconsistent with their meaning, e.g., “left” on the right side of the page), the A-X Continuous Performance Test (identifying a letter, e.g. “X,” only if certain conditions are met, for instance, being preceded by an “A”), or the Go/NoGo Task (responding to certain targets (“Go”) but ignoring distracters (“NoGo”) in a list of words, as discussed below). These and other, similar cognitive tests are known by those of ordinary skill in the art, and can be applied to a subject and the results analyzed, without undue effort.

In still another aspect of the present invention, one or more genes within the subject are determined. For example, single nucleotide polymorphisms (SNPs) may be determined in a subject that have been implicated in depression and/or response to antidepressant treatments (e.g., 5-HTT, 5-HT_{1A}), that have been implicated in depression through major discoveries (e.g., FKBP5), or that may encode processes involved in its pathophysiology (e.g., BDNF). For instance, serotonin (5-HT) may be involved in the etiology, pathophysiology, and treatment of MDD. In one set of embodiments, the present invention focuses on five genes have that have: (1) been repeatedly implicated in MDD and AD response (5-HTT, 5-HT_{1A}); (2) been recently implicated in MDD; and/or (3) not been directly implicated in the genetics of MDD but are known to code processes involved in its pathophysiology (BDNF). In other embodiments, however, one or more of these genes, and/or other genes may be determined in a subject.

Determination of the gene(s) of a subject, e.g., a SNP allele, may be performed using any suitable technique known to those of ordinary skill in the art. For example, a sample of blood (or other suitable fluid or material) may be taken from a subject, and tested to determine a SNP or other genetic marker present in the subject (e.g., a protein or other biomolecule that indicates a certain SNP is present). Those of ordinary skill in the art will be aware of suitable methods of determining a SNP (or other genetic marker) from a suitable sample of blood or other material, including saliva. A specific example is discussed in the Examples, below.

The data acquired relating to behavior, brain function, and/or genes, as discussed above, may be analyzed to determine whether a subject has or is at risk for depression, according to certain aspects of the invention. For example, the activity of the anterior cingulate cortex of the subject (or a portion thereof, such as the rostral anterior cingulate cortex), the ability of the subject to respond to negative feedback, and/or the genetic profile of the subject (e.g., the genotype of the subject with respect to genes such as 5-HT_{1A}, TPH-2, FKBP5, BDNF, and/or 5-HTTLPR) can be determined, and used to determine whether the subject has or is at risk for depression. In one set of embodiments, one or more of these determinations can be compared to a control sample (e.g., from a normal individual), and scored as positive if the subject exceeds the control sample, or exceeds the control sample by a certain amount or percentage. In some cases, the subject may be identified as being or at risk for depression if one, two, or more of the determinations are indicated as positive. In another set of embodiments, the determinations may be mathematically combined into a combined score. The determinations may be given the same or different “weights” in determining the combined score. The combined score may be compared to a control score (e.g., representing a normal individual), and the subject may have or be at risk for depression if the combined score exceeds this control value.

In some cases, the data may be analyzed using a computer. For example, data from any of the determinations discussed above (e.g., with respect to behavior, brain function, genes, etc.) may be entered into a computer, and/or the computer may be used to generate the data, e.g., via one or probes attached to a subject. For example, a computer may be programmed to conduct a negative feedback test (e.g., the Eriksen Flanker Task), and/or determine activity of the brain (e.g., using EEG techniques such as LORETA). In some cases, more than one computer may be used.

Thus, according to some aspects of the present invention, a computer and/or other system is provided able to perform any of the methods described herein, in some cases on an automated basis. As used herein, “automated” devices refer to devices that are able to operate without human direction, i.e., an automated system can perform a function during a period of time after any human has finished taking any action to promote the function, e.g. by entering instructions into a computer. Typically, automated systems can perform repetitive functions after this point in time.

Software, including code that implements embodiments of the present invention, may be stored on some type of data storage media such as a CD-ROM, DVD-ROM, tape, flash drive, or diskette, or other appropriate computer readable medium. Various embodiments of the present invention can also be implemented exclusively in hardware, or in a combination of software and hardware. For example, in one embodiment, rather than a conventional personal computer, a Programmable Logic Controller (PLC) is used. As known to those skilled in the art, PLCs are frequently used in a variety of process control applications where the expense of a general purpose computer is unnecessary. PLCs may be configured in a known manner to execute one or a variety of control programs, and are capable of receiving inputs from a user or another device and/or providing outputs to a user or another device, in a manner similar to that of a personal computer. Accordingly, although embodiments of the present invention are described in terms of a general purpose computer, it should be appreciated that the use of a general purpose computer is exemplary only, as other configurations may be used.

In some cases, a database and/or a knowledgebase may be used. For example, the database and/or a knowledgebase may store data indicative of normal individuals and/or individuals with depression, and such data may be compared, in some cases, to data from an individual subject.

Various data storage media are suitable and may include, but are not limited to, silicon integrated circuits, magnetic media, optical media, radio-frequency tags, smart cards, barcodes, and other kinds of data storage devices. In one embodiment, the data storage media includes a computer-readable medium, for example, a medium that stores information through electronic properties, magnetic properties, optical properties, etc. of the medium. Examples of computer-readable media include, but are not limited to, silicon and other semiconductor microchips or integrated circuits, bar codes, radio frequency tags or circuits, compact discs (e.g., in CD-R or CD-RW formats), digital versatile discs (e.g., in DVD+R, DVD-R, DVD+RW, or DVD-RW formats), insertable memory devices (e.g., memory cards, memory chips, memory sticks, memory plugs, etc.), “flash” memory, magnetic media (e.g., magnetic strips, magnetic tape, DATs, tape cartridges, etc.), floppy disks (e.g., 5.25 inch or 90 mm (3.5 inch) disks), optical disks, OCR readers, laser scanners, and the like. In some embodiments, the data storage component may be volatile, i.e., some power is required by the data storage media to maintain the data therein. In other embodiments, however, the data storage media is non-volatile.

The following examples are intended to illustrate certain embodiments of the present invention, but do not exemplify the full scope of the invention.

### Example 1

In this example, the study design involves open, prospective follow-up over a 12-week period of MDD subjects treated with standard doses of escitalopram, an antidepressant. Clinical response is defined in this example as a >50% change in Ham-D-17 (Hamilton Depression Rating Scale) scores from beginning to the end of trial. The main analysis correlates clinical responder status with behavioral, electrophysiological probes (EEG and ERP), and hemodynamic probes (fMRI) of rostral ACC (anterior cingulate cortex) function as well as with allelic variation in five candidate genes (5-HTTLPR, 5-HT_{1A}, TPH-2, FKBP5, and BDNF). Secondary analyses will assess links between SNPs (single nucleotide polymorphisms) of these genes and behavioral/physiological measures of affective disturbance. A total sample of 85 MDD subjects will be studied using an integration of laboratory-based measures of symptom profiles, high-density EEG/ERP, fMRI, and genotyping.

Subject Recruitment. A minimum of 85 depressed subjects will be enrolled in a depression clinical and research program involving a 2-week, open-label treatment with escitalopram. The subjects will be enrolled over a 28 months period, enrolling approximately 3 subjects/month. With a conservative estimate of a 15% drop-out rate, approximately 72 will complete 12 weeks of open-label treatment with escitalopram. Considering a response rate of 55%, about 40 subjects will be treatment responders, and about 32 subjects will be nonresponders at the end of treatment. Subjects having MDD, diagnosed with the Structured Clinical Interview for DSM-IV-Axis I disorders, and who have baseline scores on the 17-item Hamilton Depression Rating Scale of 16 or greater will be enrolled. Enrolled MDD subjects will satisfy the Inclusion and Exclusion criteria summarized in Table 3.

Treatment. Subjects screened for the study and found to be eligible will return for a baseline visit after one week, during which no psychotropic medication will be allowed. The baseline visit and the physiology sessions (ERP, fMRI) will occur after this interval has passed. Patients with a decrease in Ham-D-17 score of >25% from screen to the baseline visit will be excluded. Enrolled patients will begin a 12-week treatment with escitalopram. Patients will be started on escitalopram 10 mg/day for 4 weeks. All patients will be instructed to return their medications at each visit, and a pill count will be done to corroborate the drug record.

Dose selection. The protocol in Table 1 will utilize the dose recommended by the manufacturer and approved by the FDA for depression (10-20 mg/day). For the first 4 weeks, the subjects will take 1 tablet escitalopram 10 mg/day. For the next 4 weeks; the treating clinician will have the option to increase the dose to 20 mg/day, if tolerated, for patients determined to be nonresponders. After 12 weeks, all nonresponders and patients who drop out from the study will be offered 3 months of open treatment with another antidepressant. Responders to escitalopram will be offered 3 months of follow-up care.

Rationale for selecting escitalopram. There are at least two reasons for choosing escitalopram. First, SSRIs (selective serotonin reuptake inhibitors) are considered the standard, first-line treatment for depression, and escitalopram, an SSRI, is one of the top three most highly prescribed SSRIs. Second, at 10 mg/day, escitalopram has been found to have comparable attrition rates as placebo, which may minimize the risk of patient attrition due to adverse drug effects.

TABLE 1

| Week 0 | Weeks 1-4 | Weeks 5-12 |
|---|---|---|
| Enter | Escitalopram, 10 mg/d | Responder: Continue escitalopram |
| | | at 10 mg/d |
| | | Non-responder: Increase escitalopram |
| | | to 20 mg/d |

Frequency of Visits. Subjects will be assessed according to the following schedule:

TABLE 2

| | | Visit | | Visit | | Visit | |
|---|---|---|---|---|---|---|---|
| Screen | Baseline | 1 | Visit 2 | 3 | Visit 4 | 5 | Visit 6 |
| Week-1 | Week 0 | Week | Week 4 | Week | Week 8 | Week | Week 12 |
| | fMRI, | 2 | | 6 | | 10 | (endpoint) |
| | EEG | | | | | | |

TABLE 3
Inclusion Criteria:

| | |
|---|---|
| 1) | DSM-IV diagnostic criteria for MDD (diagnosed with the use of SCID). |
| 2) | Written informed consent. |
| 3) | Both genders and all ethnic origins, age between 18 and 65. |
| 4) | A baseline Hamilton-D17 score of >16. |
| 5) | Right-handed. |

Exclusion Criteria:

| | |
|---|---|
| 1) | Subjects with suicidal ideation where outpatient treatment is determined unsafe |
| | by the study clinician. These patients will be immediately referred to appropriate |
| | clinical treatment. |
| 2) | Pregnant women or women of childbearing potential who are not using a medically |
| | accepted means of contraception (defined as oral contraceptive pill or implant, |
| | condom, diaphragm, spermicide, IUD, s/p tubal ligation, partner with vasectomy). |
| 3) | Serious or unstable medical illness, including cardiovascular, hepatic, renal, |
| | respiratory, endocrine, neurologic or hematologic disease. |
| 4) | History of seizure disorder. |
| 5) | History or current diagnosis of the following DSM-IV psychiatric illness: organic |
| | mental disorder, schizophrenia, schizoaffective disorder, delusional disorder, |
| | psychotic disorders not otherwise specified, bipolar disorder, patients with mood |
| | congruent or mood incongruent psychotic features, patients with substance |
| | dependence disorders, including alcohol, active within the last 12 months. |
| 6) | History or current diagnosis of dementia, or a score of <26 on the Mini Mental |
| | Status Examination at the screening visit. |
| 7) | History of multiple adverse drug reactions or allergy to the study drugs. |
| 8) | Patients with mood congruent or mood incongruent psychotic features. |
| 9) | Current use of other psychotropic drugs. |
| 10) | Clinical or laboratory evidence of hypothyroidism. |
| 11) | Patients who have failed to respond during the course of their current major |
| | depressive episode to at least one adequate antidepressant trial, defined as six |
| | weeks or more of treatment with escitalopram 10 mg/day (or citalopram 20 |
| | mg/day). |
| 12) | Patients with lifetime electroconvulsive therapy (ECT). |
| 13) | History of intolerance to escitalopram. |
| 14) | Subjects taking antidepressants at the time of their screening visit will be enrolled |
| | only if they are willing (after discussing with their prescribing clinician), and the |
| | study clinician determines that it is clinically appropriate for them to discontinue |
| | their current antidepressant for a period greater than five half-lives of their current |
| | medication (but no longer than 7 days). For these subjects, the baseline visit and |
| | the first physiology session will only occur after the 7 day interval has passed. |
| 15) | Failure to meet standard MRI safety requirements. |

Efficacy Data. The following instruments will be administered at the Screen and Baseline time points: Structured Clinical Interview for DSM-IV (SCID) (screen visit only); Mini Mental Status Examination (screen visit only); 28-Item Hamilton Rating Scale for Depression (HAM-D 28-item); and Clinical Global Impressions-Severity (CGI-S) and Improvement (CGI-1). The following instruments will be administered at the Screen, and Visits 1 and 6 time points: Kellner's Symptom Questionnaire; and Atypical Depression Diagnostic Scale (ADDS).

Safety Data. The following laboratory tests will be performed at the Screen visit: Complete blood count; urinalysis; comprehensive metabolic panel (serum concentrations of electrolytes, BUN (blood urea nitrogen), creatinine, SGOT (serum glutamic oxaloacetic transaminase), SGPT (serum glutamate pyruvate transaminase), CPK (creatine phosphokinase), alkaline phosphatase, total bilirubin, albumin, total protein, and glucose); TSH (thyroid-stimulating hormone); and EKG (electrocardiogram). A physical exam will be performed at the Screening visit and at study end. Vital signs will be recorded at each visit.

Adverse Events. Adverse effects will be monitored and documented throughout the study. Documentation of the presence of any side-effect or adverse event will be completed by the treating psychiatrists at every visit.

Concomitant Therapy. All concomitant medications taken during the study will be recorded. Any prescription or over-the-counter medication not excluded by the protocol will be allowed (e.g., aspirin, cold preparations). Subjects requiring excluded drugs (e.g., other antidepressants, benzodiazepine sedatives, antipsychotics, psychostimulants, and mood stabilizing agents) will be discontinued from the study.

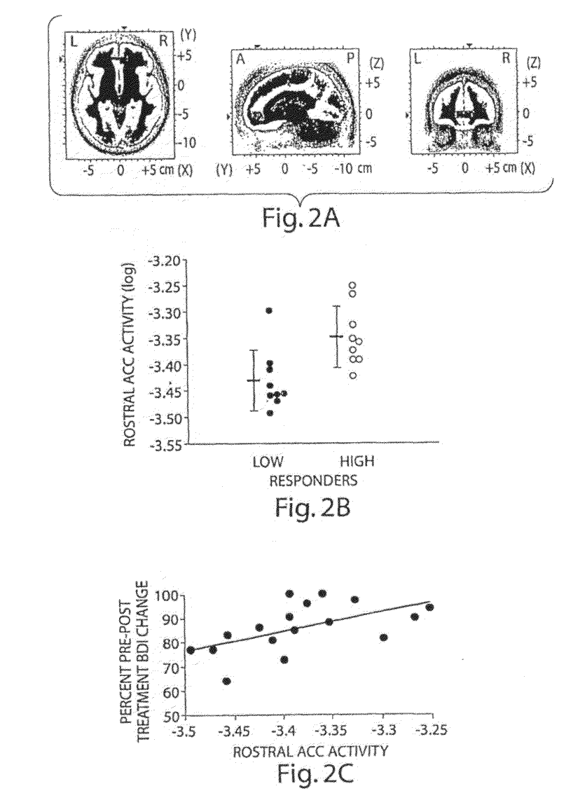

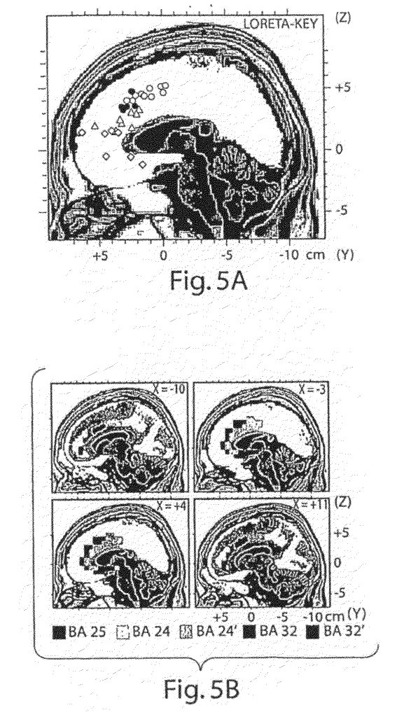

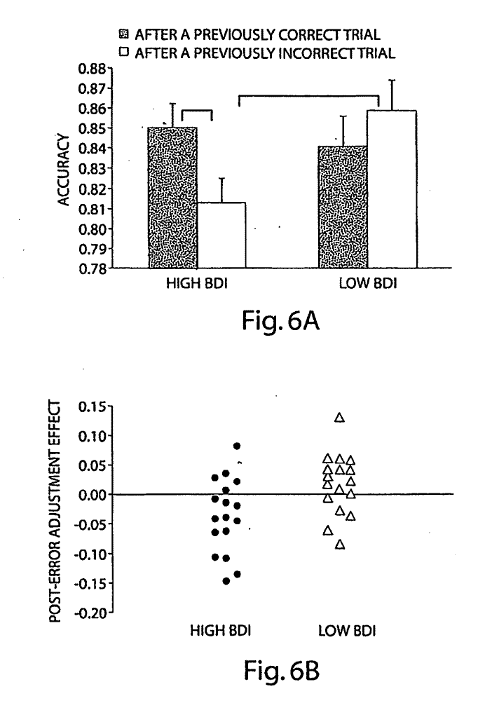

Sample Size and Power Calculations. The sample size was estimated after considering effect sizes obtained in recent pilot studies. Effect sizes were calculated using an α (alpha) level of 0.05 (2-tailed) for detecting differences between responders and nonresponders to escitalopram. Based on these calculations, allowing for 15% data loss, and considering 55% treatment response rate, this study will follow 85 subjects (estimated treatment responders: 40 subjects; estimated nonresponders: 32 subjects). In a preliminary study outlined above, responders and nonresponders differed in rostral ACC activity before nortriptyline treatment (Cohen's d=1.33). When considering the outcome data as continuous variables, a relation between rostral ACC activity and % BDI (Beck Depression Inventory) change was found (r=0.57). See FIG. 2 and FIG. 5. FIG. 7 shows a correlation between the post-error adjustment effects and the gamma current density residuals in the affective ACC subdivision (Brodmann Area BA 24). These effect sizes were notable, particularly considering that this study involved an open-label treatment without randomization. Thus, although this design likely induced significant placebo effects, the link between pre-treatment rostral ACC activity and treatment response remained robust. It is expected that the sample of 85 MDD subjects will allow the identification of behavioral and electrophysiological (and hemodynamic) surrogate markers of non-response. In fact, for both categorical and continuous variables, the power to detect EEG/ERP differences between responders and nonresponders with the proposed sample of 72 is >0.99.

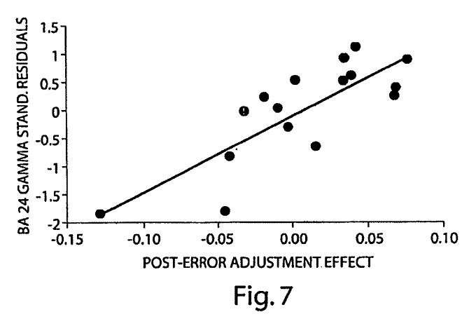

In a study using the Eriksen Flanker Task, it was found that subclinically depressed and control subjects differed in post-error adjustment effects (d=0.95) and resting activity within the rostral ACC (d=1.01). Considering a mean effect size of 0.98, a total of 72 subjects was linked to a power >0.99 of detecting behavioral and EEG/ERP differences of rostral ACC function between responders and nonresponders. To assess if the proposed sample would allow the detection of group differences when considering various estimates of placebo effect (“PE”), simulations using G*Power (see, e.g., Erdfelder, E., Faul, F., & Buchner, A. (1996). GPOWER: A general power analysis program. Behavior Research Methods, Instruments, & Computers, 28, 1-11) were run (FIG. 8). These findings revealed that, at least for the EEG and behavioral ACC probes (mean d: 1.10), a final sample of 44 (PE: 38.9%), 36 (PE: 50%) and 30 (PE: 58.3%) still provided a power >0.82.

Power for genetic analyses was calculated using the Genetic Power Calculator (Purcell S, Cherny S S, Sham P C. (2003) Genetic Power Calculator: design of linkage and association genetic mapping studies of complex traits. Bioinformatics, 19(1):149-150) for the anticipated completer sample (40 responders, 32 non-responders). Based on prior studies, a multiplicative genotypic relative risk (GRR) model (see, e.g., Schaid, D. J. and Sommer, S. S. (1993) Genotype relative risks: methods for design and analysis of candidate-gene association studies. Am. J. Hum. Genet. 53, 1114-26) was assumed, in which the marker allele is the risk allele or is in complete LD with the risk allele. It was determined that the power for possible combinations of a range of allele frequencies (0.1-0.5) and GRRs (1.25-2.0) was consistent with those reported in prior studies with these SNPs. Power is appropriate as long as GRR>1.5 (see Table 4). For the smallest effects, power might be limited.

TABLE 4

| GRR | Power |
|---|---|
| 1.25 | 16-31% |
| 1.5 | 75-77% |
| 1.75 | 95-96% |
| 2.0 | 99% |

These studies will show that, compared to eventual responders, nonresponders to escitalopram will show significantly lower resting theta activity in the rostral ACC before treatment; lower post-error adaptation effects; higher ERN; lower Pe; lower fMRI signal in rostral ACC regions during affective conflict, i.e., NoGo trials; and/or higher frequency of the s variant of the polymorphism in the 5-HTTLPR, the G variant at the C(−1019)G polymorphism of the 5-HT1A gene, the mutant (1463A) allele of the TPH-2 gene, the CC homozygotes for rs1360780 polymorphism of the FKBP5 gene, and/or the met allele of the val66met BDNF gene.

In addition, these studies will determine if increased ERN, impaired affective conflict monitoring abilities, decreased rostral ACC activation, and/or dysfunctional frontocingulate connectivity may be associated with higher frequency of the short allele of the 5-HTTLPR and/or the met allele of the val66met BDNF gene.

Procedure. After obtaining consent, subjects will be administered the SCID-I/P (Structured Clinical Interview for DSM-IV). Patients meeting criteria for MDD and fulfilling the inclusion criteria (Table 3) will be scheduled for both fMRI and ERP sessions, which will be counterbalanced across subjects. These sessions will occur after the washout period and before treatment, and will provide pre-treatment behavioral, EEG/ERP, and fMRI data. At both sessions the Apathy Evaluation Scale will be administered.

Behavioral and EEG Session: Eriksen Flanker Task. A speeded version of the Eriksen task, known to elicit response conflict and a high error rate, will be used. Subjects will be instructed to respond as fast as possible to a target arrow presented for 30 ms in the center of the screen. When the target arrow points to the right, a right button press will be required (and vice versa). To induce errors that will be critical for the analyses, the target arrow will be preceded by task-irrelevant flankers (arrows pointing to the left or to the right) presented for 100 ms above or below the center of the screen. In 50% of the trials, the flanker and target arrows will point in the opposite direction, thus causing conflict (“incompatible trials”); in the remaining 50% of the trials, they will point in the same direction (“compatible trials”). Correct and incorrect responses will be followed by positive (a schematic smiling face) and negative (a frowning face) feedback, respectively (presented for 500 ms).

fMRI Session: Affective Go/NoGo Task. Briefly, 24 task blocks will be interspersed with 24 rest blocks. Before each block, subjects will be given instructions to respond to certain targets (“Go”) but ignore distractors (“NoGo”). In the main conditions, subjects will respond to happy, sad, or neutral targets. In a control condition, words will be neutral, and the targets will be defined on the basis of font (italic vs. plain text). Words will be selected from the Affective Norms for English Words list, and will be matched for length and frequency. Emotional words will be also matched for valence intensity and arousal. The blocks with target (1) and distractor (D) stimuli will be: (1) happy (T), sad (D); (2) happy (T), neutral (D); (3) sad (T), happy (D); (4) sad (T), neutral (D); (5) neutral (T), sad (D); (6): neutral (T), happy (D); (7) italic text (T), plain text (D); and (8) plain text (T), italic text (D). In each block, 10 targets and 10 distractors will be presented in a randomized order. Each word will be presented for 300 ms, followed by an ISI of 900 ms, upon which the next word will be presented. Each 20-word block will last 24 s and will be preceded by a 24-s rest block.

Rationale. Given established links between rostral ACC function and treatment response and theories emphasizing frontocingulate circuits in MDD, tasks were needed that reliably activate the rostral ACC, and/or engaged PFC regions.

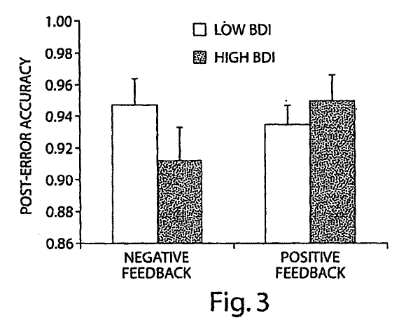

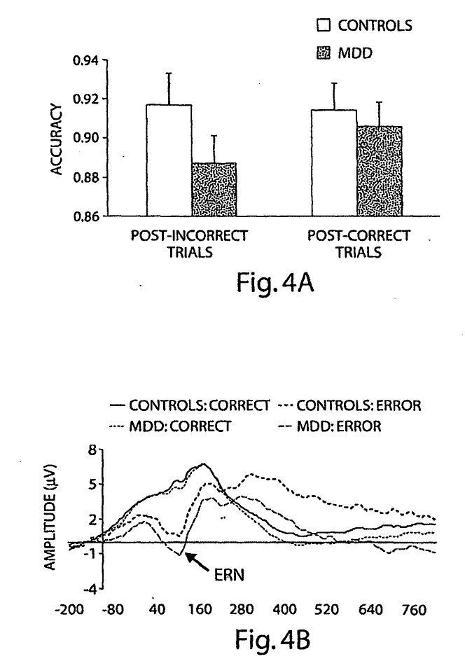

Eriksen Flanker Task. Prior studies using this task have shown that performance can be decomposed into different subcomponents, including behavioral adjustments during or after high-conflict (incompatible) trials, as well as after errors. For example, subjects typically slow down their RT (response time) and improve their accuracy on trials following errors, suggesting that they utilize errors to monitor and improve their performance. See FIGS. 3 and 4. Experiments have also shown that dorsal and rostral ACC regions are primarily recruited during conflict monitoring and error detection, respectively (FIG. 5). In FIG. 5A, circles show activation during conflict monitoring, triangles show activation during error commission, and diamonds show rostral ACC linked to treatment response in MDD. FIG. 5B shows the location of various ACC regions. FIG. 5C shows mean gamma wave activity within five general ACC regions for low and high BDI subjects. The regions are identified by their Brodmann numbers. FIG. 5D shows mean gamma wave activity within the affective and cognitive ACC subdivisions. Importantly, it was found that dysphoric subjects showed reduced accuracy immediately after committing an error, and resting rostra ACC activity predicted individual differences in post-error behavioral adjustments.

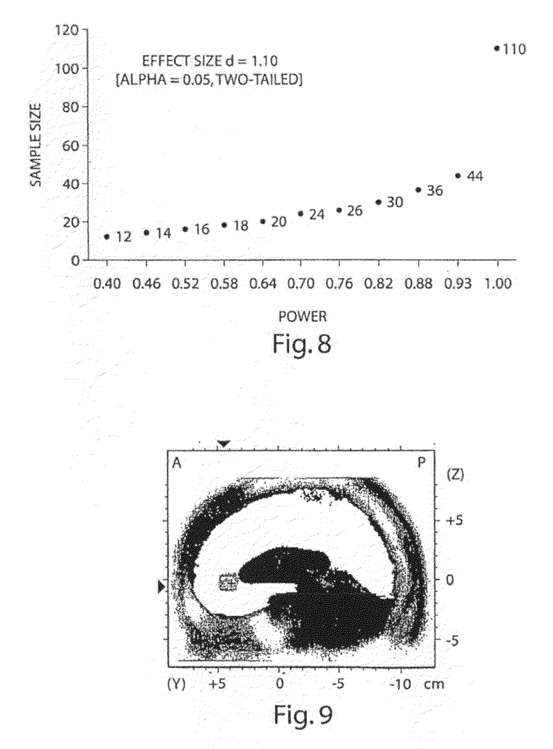

Affective Go/NoGo Task. This task was chosen because it allows the assessment of executive function and putative mood-congruent biases that may differentially influence conflict monitoring abilities, impairments in affective monitoring have been implicated in MDD, and the maximal locus of ACC activation in the affective Go/NoGo overlaps with the rostral ACC regions implicated in treatment response (FIG. 9).

ERP data. 128-channel EEG will be recorded using the Geodesic Sensor Net system (EGI, Oregon), where EEG electrodes are arrayed in a regular distribution across the head (inter-sensor distance: ˜3 cm). Stimulus presentation will be controlled by “E-Prime for Net Station” (Psychology Software Tools, Inc, Pittsburgh, Pa.), a software suite designed for running experiments in conjunction with the EGI system.

fMRI data. The fMRI session will take place on a 1.5 T Siemens MRI scanner. Subjects will be escorted to the scanner room, provided with ear protection, and positioned in the scanner. After collection of anatomical images (3D gradient-recalled echo with spoiler gradient sequence; 1-mm coronal slices) that will be used for normalization of functional data and ERP-fMRI coregistration, gradient echo T2*-weighted echoplanar images (EPI) will be acquired. To improve signal in regions affected by susceptibility artifacts, EPI images will be acquired using image tilting and z-shimming with the following parameters: TR/TE: 2500/35 ms; FOV: 200 mm; matrix: 64×64; number of slices: 36; in-plane resolution: 3 mm (2-mm thick slices, 1-mm gap); slice orientation: oblique (30° from the AC-PC line; rostral>caudal).

DNA Extraction. Blood samples (30 ml) will be collected from each subject and coded with a study ID. Genomic DNA will be extracted using the Puregene DNA Purification Kit from Gentra Systems within 2-5 days of the sample collection and stored in the laboratory.

Sample Tracking. Data (date, site of origin, study, clinical investigator's code, gender, race/ethnicity) for received DNA samples will be entered into a computer. An ordinal ID number is assigned at this time and is linked to the subject's study ID with a barcoding system. In addition to tracking samples, the computer can be used to design and layout large-scale genotyping experiments.

Genotyping Methods. In order to limit the problem of multiple testing, a hypothesis-driven approach is used, limiting analyses to five loci selected based on prior evidence implicating them in antidepressant response (5-HTT, TPH-2, FKBP5) and/or MDD (5-HTT, TPH-2, FKBP5, 5-HT_{1A}, BDNF) (Table 5).

TABLE 5

| | Locus Name; | | Polymorphism | Relevant |
|---|---|---|---|---|
| Gene | Location | Function | (Approx MAF*) | phenotypes implicated |
| Brain-derived | BDNF; 11p13 | Neuronal growth | Val66Met (rs6265) | AD action; MD; BD |
| neurotrophic | | and development | (.28) | |
| factor | | | GT(n) microsatellite | |
| FK506 binding | FKBP5; | Glucocorticoid- | rs3800373 (.36) | AD response; |
| protein 5 | 6p21.3-21.2 | regulating co- | rs1360780 (.25) | recurrent MD |
| | | chaperone | rs4713916 (.24) | |
| Serotonin | HTR1A; | Receptor mediating | C-1019G (rs6295) | MD |
| 1A receptor | 5q11.2-q13 | serotonergic effects | (.33) | |
| Serotonin | SLC6A4; | Serotonin | 5HTTLPR (.45) | SSRI response; MD |
| transporter | 17q11.1-q12 | reuptake | | |
| Tryptophan | TPH2; | Neuronal | G1463A (0.01-0.10) | SSRI response; MD |
| hydroxylase | 12q21.1 | serotonin | rs1386494 (.12) | |
| | | synthesis | | |

Note:
MD = major depression; BP = bipolar disorder; AD = Antidepressant
*MAF = approximate minor allele frequency for diallelic variants based on prior studies or public databases

The analysis has also been limited to polymorphisms that have previously been associated with these phenotypes. 20 unlinked (null) microsatellite markers will also be genotyped to permit evaluation of population stratification. SNP genotyping will be performed using the Sequenom MassArray system. To minimize reagent cost, individual genotyping reactions will be performed in multiplex format. SNPs are amplified in multiplex PCR reactions consisting of four loci each. The volume of the PCR reaction is kept small (5 microliters) to minimize reagent costs and DNA consumed (2.5-5 ng/SNP). Primers are designed using SpectroDESIGNER software to have a midpoint of thermal denaturation between 56° C. to 60° C. with a mass range between 5000 Da to 8000 Da. Genotyping will be performed in multiplex reactions in 384-well plates. For each assay, 4 duplicate samples and 4 blank samples will be included. SNPs will be used for association analyses if they meet the following criteria: 1) >90% of attempted genotypes for any SNP are successful; 2) alleles are in Hardy-Weinberg equilibrium; and 3) agreement between all duplicates and no more than 1 of 4 blanks with genotypes. Genotyping of microsatellites and the 5-HTTLPR will be performed using the Applied Biosystems 3730 DNA Analyzer.

Population Stratification. The 20 unlinked (null) microsatellite markers will be genotyped to permit evaluation of population stratification.

Efficacy and Treatment Outcome Data. The primary measure of effectiveness will be reduction on blindly rated HRSD-17 total scores over the course of 12 weeks of acute treatment. HRSD-17 change scores will be analyzed as a continuous and dichotomous variable, with response defined as >50% reduction in HRSD-17 scores. Remission will be defined as a HRSD-17 score equal or less than 7.

Resting EEG Data. Previous work has implicated the theta band in treatment outcomes in MDD, as well as functional coupling between the theta and gamma band. Intracerebral sources (current density) of theta (6.5-8 Hz) and gamma (36.5-44 Hz) activity will be thus computed using Low Resolution Electromagnetic Tomography, as previously described. Pretreatment resting EEG data will be analyzed using both dichotomous and continuous outcome variables.

Behavioral Data-Eriksen Flanker Task. In addition to analyzing overall RT and accuracy, compatibility (Eriksen), post-error adjustment, and conflict-adaptation effects will be computed, since these variables have been linked to conflict monitoring/dorsal ACC (Eriksen/Gratton effect) and post-error behavioral adjustments/rostral ACC (Laming effect). The Compatibility effect will be computed as: [RT_{Incompatible trials}−RT_{Compatible trials}] and [Accuracy_{Compatible trials}−Accuracy_{Incompatible trials}]. The post-error adjustment effect will be computed as: [RT_{After incorrect trials}−RT_{After correct trials}] and [Accuracy_{After incorrect trials}−Accuracy_{After correct trials}]. The Conflict-adaptation effect will be computed as: [RT_{Incompatible trials following compatible trials}−RT_{Incompatible trials following incompatible trials}] and (Accuracy_{Incompatible trials following incompatible trials}−Accuracy_{Incompatible trials following compatible trials}]. Group (Responders vs. Nonresponders)×Condition (e.g., accuracy after incorrect vs. correct trials) ANOVA will be performed, and the interaction will be formally tested.

Behavioral Data-Affective Go/NoGo Task. Besides analyzing overall RT and accuracy, signal-detection analyses will be used to calculate response bias and sensitivity scores based on hit rates and false alarms.

ERP Data (Eriksen Flanker Task). After gain and zero calibration, data will be analyzed with NetStation 3.0 software. Channels with corrupted signals will be interpolated using a spline interpolation method. After off-line automatic artifact rejections, ERPs will be computed covering 1024 ms and time-locked to the onset of target arrow and subject's response (100-ms pre-stimulus baseline). ERP will then be baseline-corrected, lowpass filtered at 35 Hz (12 dB/octave roll-off), and re-referenced to the average reference. As in past studies using similar paradigms, the ERN will be defined as the highest negative peak (peak-to-baseline difference) over frontocingulate leads (e.g., FCz) within a time window starting 20 ms before the response and 130 ms post-response. The ERN will be calculated as the amplitude difference after erroneous minus correct responses. Pe will be defined as the highest positive peak within a time window from 130-450 ms. In addition to traditional ERP waveform analyses, space-oriented brain electric field analysis will be utilized. To increase spatial sensitivity of scalp ERP analyses, t-tests will be run at each sensor contrasting the various conditions. Statistical Non-Parametric Mapping will be used to correct for Type I error. For periods showing significant scalp findings, the cortical 3-D distribution of current density will be computed with LORETA. For these studies, a version based on a three-shell spherical head model and EEG electrode coordinates derived from cross-registrations between spherical and realistic head geometry will be used. The head model is registered to a standardized stereotactic space (Montreal Neurologic Institute, MNI305). The source solution space is limited to cortical gray matter and hippocampi according to MNI Probability Atlases (voxel: 7 mm³). Whole-brain analyses using voxelwise t-tests will then examine differences between groups or conditions. Monte-Carlo permutations will be used to correct for Type I error. The Structure-Probability Maps atlas will be used to label regions and Brodmann areas with significant differences between conditions or groups. For the ERN, a Group×Condition interaction is expected, due to higher activation in treatment nonresponders in error and post-error trials than responders. For the Pe, a Group×Condition interaction is expected, due to lower activation in treatment nonresponders in error and post-error trials than responders.

For both the behavioral and physiological data, additional analyses will be conducted to assess whether apathy, which might be present in some MDD subjects, modulates the primary findings. A hierarchical regression will be used instead of analyses of covariance (ANCOVA) to prevent confounds caused by the covariate (AES score) correlating with the independent variable (Group). These analyses will test whether Group uniquely predicts the hypothesized findings after controlling for AES score and the Group×AES score interaction.

fMRI Data (Affective Go/NoGo Task). After slice-time correction, motion correction, and detrending to eliminate drift effects, fMRI data will be spatially smoothed with a Gaussian filter (FWHM: 5 mm³) to take into account anatomical individual variations. Preprocessing of MRI data will be performed with “fiswidgets,” a platform for various analysis packages (e.g., AIR Automated Image Registration, see, e.g., Woods, R. P., Grafton, S. T., Watson, J. D. G., Sicotte, N. L., and Mazziotta, J. C. (1998). Automated image-registration. II. Intersubject validation of linear and nonlinear models. Journal of Computer Assisted Tomography, 22(1):153-165., AFNI (Analysis of Functional NeuroImages, an open source computer program)). To control for the influence of task performance, hemodynamic responses from error and no-response trials will be discarded. Using SPM2 (Statistical Parametric Mapping, available for MatLab), a random-effects model will be run to allow population-based inferences. For each subject, one mean image per condition will be generated, and images will then be combined in a series of linear contrasts to assess group effects. The following contrasts will be performed: Emotional vs. Neutral Targets; Happy vs. Sad Targets; Happy vs. Sad Distracters; Semantic vs. Orthographic Conditions. The semantic vs. orthographic comparison will serve as control contrast, in which no differences between the responders and nonresponders are expected. Voxel-by-voxel ANOVAs with Group (Responders, Nonresponders), Trial (Go vs. NoGo trials), and Valence (Negative, Neutral. Positive) as factors will be run. The Group×Trial×Valence interaction will likely be significant, due to lower activation in Nonresponders in the rostral ACC during NoGo trials than Responders, particularly with sad distractors.

To investigate whether hemodynamic differences between responders and nonresponders might be confounded by volume differences, an automated method will be used to measure cortical thickness. This approach can be utilized to measure cortical thickness within the ventral, rostral, and dorsal ACC. Variables extracted from this procedure will be used as a covariate to assess the degree to which cortical thickness at the locus of peak activation is associated with group differences in activation.

Cross-modal Analyses of ERP and fMRI Data. Six steps will be performed for cross-modal analyses. First, EPI images will be smoothed with a 6 mm³ Gaussian filter to approximate the spatial resolution of LORETA (˜10 mm). As in recent ERP-fMRI and EEG-PET studies, current density and fMRI data will be spatially co-registered to the MNI305 template using SPAMALIZE software (a software package used to analyze and display image data). Cortical clusters showing task-related modulation in the fMRI data will be identified, and their coordinates will be used to extract current density for the ERP data. Unfolding of activation in these regions will be investigated using code that displays current density within user-specified ROI (region of interest) as a function of time. Task-dependent functional connectivity analyses will be performed on the LORETA data to test whether responders and nonresponders differ in neural pathways subserving conflict monitoring. Analyses of functional connectivity will be particularly interesting considering that allelic variants of the BDNF gene will be investigated, BDNF has been implicated in synaptic plasticity, and neural plasticity has been involved in treatment mechanisms in MDD. To this end, correlations will be run between the averaged current density in a given ROI and activity at each voxel. At each voxel, a Fisher test will be computed to assess whether the two groups differ in correlation patterns. Further analyses will assess whether functional connectivity unfolds differently for responders and nonresponders. Thus, current density in user-specified regions will be correlated with activity at each other voxel throughout time. These analyses will allow testing of, for example, whether dysfunctional ACC activation in response to a given condition will predict DLPFC activation at a later step of the information processing flow.

Integration Across Levels of Analyses (Genes, Brain, Behavior). One goal of the present study is to identify prospectively those MDD subjects who will later show a particular response to treatment (in this case, to SSRIs) based on pretreatment measures of brain and cognitive function. In the present study, an analogous logistic regression approach will be used to develop multivariable models (“algorithms”) aimed to estimate the probability of treatment response. Specifically, neurophysiological (e.g., resting rostral EEG activity), behavioral (e.g., post-error behavioral adjustment), and genetic (e.g., TPH-2) variables showing pre-treatment differences between responders and nonresponders will be entered as independent variables. Assuming an additive model of gene action, a predictor variable 0, 1, 2 will be used (dosage effect of having 0, 1 or 2 alleles) for the genetic information. Using a final model with a classification cutoff of 0.5, forward stepwise regression will be used to identify variables with the strongest predictive value. For the logistic regression model, Nagelkerke's R² will be used to test the strength of association between the independent and dependent variables. The chi-square model will be used to assess the improvement in fit when the independent variables are in the model vs. the null model. Finally, logistic regression coefficients will be assessed using log-likelihood ratio tests to assess the significance of the individual variable, while holding constant all other independent variables.

To control for Type I error, Statistical Non-Parametric Mapping (SnPM) will be used for fMRI, scalp ERP, and LORETA data. Without assuming any distribution, SnPM uses permutation tests to estimate the false-positive rate under the Null Hypothesis of no group or condition differences. For scalp ERP data, code available in the LORETA package will be utilized to compute the exact probability that groups or conditions differ in scalp topography within user-specified ERP windows. A randomization procedure using the tₘₐₓ approach will be used for LORETA data. For the fMRI data, a similar procedure implemented in SPM2 will be used to achieve a mapwise significance level of p<0.05.

Another statistical approach that may be used is as follows. A logistic regression approach can be used to develop multivariable models (“algorithms”) aimed at identifying predictors of treatment response. First, a set of candidate predictors of clinical (e.g., numbers of prior episodes), neurophysiological (e.g., resting rostral EEG activity), behavioral (e.g., post-error behavioral adjustment), genetic (e.f., 5-HTTLPR), and fMRI variables will be identified, showing significant pre-treatment differences by the univariate comparisons between responders vs. nonresponders as well as remitters vs. non-remitters. If necessary, proper transformations or creation of quartile-based ordinal scale variables will be considered for variables with skewed distributions. Also, potential non-linear relationships of each variable with HAMD-17 total scores prior to group dichotomization will be examined. If necessary, the variables that indicate non-linear relationships will be considered for proper transformations. The variables with a p-value<0.05 will be entered as the candidate predictor variables in the multivariate logistic modeling. The final model will be determined via forward stepwise selection procedure, with a classification cutoff of 0.5, to include only the significant predictors at 5% alpha level as well as to identify variables with the strongest predictive value. The log-likelihood ratio chi-square will be evaluated to assess the improvement in fit when the predictor variables, are in the model vs. the null model, and Nagelkerke's R2 will be used to test the strength of association between the treatment outcome and the predictor variables.

### Example 2

This example illustrates a novel assay for prediction of treatment response in depression.

In order to determine the specificity/sensitivity values of the rostral ACC for predicting treatment response in depression, ROC analyses and discriminant analyses was performed, as well as logistic modeling on 28-channel EEG data. (See Pizzagalli, D A, et al. (2001), “Anterior cingulate activity as a predictor of degree of treatment response in major depression: Evidence from brain electrical tomography analysis.” Am J Psychiatry 158: 405-415.)

Sixteen of the 18 subjects in this study could be considered as “clinical responders”, since their BDI dropped by more than 63% from the pre- to the post-treatment assessment (the remaining two are “non-responders”). The rostral ACC activity predicted the degree of treatment response in spite of the fact that 16 of the 18 subjects were true responders, suggesting that the rostral ACC measure can capture small variations in clinical outcome, and the difference between true clinical “responders” and “non-responders” will likely be even larger.

In the present study, in the ROC analyses, using a specific cutoff of rostral ACC activity, 88.9% of eventual “high” responders would be correctly identified as such, and 11.1% of eventual “low” responders would be incorrectly identified as “high” responders. The area under the curve was 0.889.

In the discriminant analyses, 8 of the 9 “high” responders were correctly classified as such, whereas 6 of the 9 “low” responders were correctly identified as “low” responders. Accordingly, 77.8% of original grouped cases were correctly classified.

Since there were only 18 subjects in this sample, an internal validation was also performed, where 30 logistic models were fitted, each involving 16 randomly chosen observations, allowing empirical distributions of sensitivity and specificity over 30 repeats to be drawn. The average sensitivity over the 30 repeated fits was 92.4% and the average specificity was 77.4%.

Another goal of these analyses was to compare the predictive value of rostral ACC activity (as inferred by LORETA) vs. traditional scalp power spectrum analyses with respect to treatment response. The LORETA and scalp FFT data were derived from the same set of EEG data; were processed in a conceptually equivalent way; and both focused on the theta band (please see below for a summary of the scalp spectral analyses).

For any effects found in the LORETA analyses, conventional scalp power analyses were conducted for the corresponding bands using the same non-overlapping EEG epochs. For these analyses, a Fast Hartley Transform and a Hamming window were employed. After re-referencing the data to the average reference, power density (μV²/Hz) was computed by summing power values across each 0.5 Hz and dividing by the number of bins. Subsequently, for each channel, mean power density was computed (weighted by the number of artifact-free epochs), log-transformed, and finally whole-head residualized using the mean band power across the 28 electrodes.

Among the 15 frontocentral sites tested (F7/8; FC7/8; FP1/2; C3/4; Cz; F3/4; FC3/4; FPz; Fz), only Cz showed significant differences between eventual responders and non-responders. Compared to non-responders, responders showed significantly higher theta power at Cz (p=0.026; effect size: d=1.16). No significant differences emerged when comparing eventual responders and control subjects (p=0.19; effect size: d=0.58). When considering the measure of rostral ACC activity, the effect sizes were responders vs. non-responders (p=0.008; effect size: d=1.43); responders vs. controls (p=0.013; effect size: d=1.14).

The ROC/Discriminant analyses using scalp theta power at Cz to classify responders/non-responders were repeated. In the ROC analyses, the area under the curve was 0.778 (vs. 0.889 when using rostal ACC activity). The same level of correct classification of eventual “high” responders (88.9%) could be achieved through a substantially higher misclassification on non-responders (55.6% vs. 11.1% when using rostral ACC activity). For the present sample, no cutoff value for theta power at Cz gave a satisfactory balance between sensitivity and specificity.

In the discriminant analyses, when considering theta power at Cz, 7 of the 9 “high” responders were correctly classified as such, whereas only 5 of the 9 “low” responders were correctly identified as “low” responders. Accordingly, 66.7% of original grouped cases were correctly classified (vs. 77.8 when using rostral ACC activity).

In sum, compared to “traditional” scalp spectral analyses, the above described method, as discussed herein, was associated with higher statistical power and a more precise classification of eventual responders (see Table 6).

TABLE 6

| | Scalp power | |
|---|---|---|
| | spectrum analyses | Rostral ACC activity |
| | (theta power at Cz) | (as estimated by LORETA) |
| Effect sizes | | |
| Responders vs. | Cohen's d = 1.16 | Cohen's d = 1.43 |
| Non-responders | (p = .026) | (p = .008) |
| Responders vs. | Cohen's d = 0.58 | Cohen's d = 1.14 |
| Controls | (p = .19) | (p = .013) |
| ROC analyses | | |
| Area Under the Curve | 0.778 | 0.889 |
| Discriminant Analyses | | |
| Sensitivity | 88.9% | 88.9% |
| 1 - Specificity | 55.6% | 11.1% |
| Correct classification | 7/9 | 8/9 |
| Responders | | |
| Correct classification | 5/9 | 6/9 |
| Non-Responders | | |

Although the invention has been described with respect to various embodiments, it should be realized this invention is also capable of a wide variety of further and other embodiments. Accordingly, while several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A method for predicting the success or failure of a given treatment for treating individuals diagnosed with or predisposed toward a psychiatric disorder, comprising analyzing behavior of said individual, wherein said behavior analysis is directed toward rostral ACC function of said individual.

2-4. (canceled)

5. The method of any of claims 1, wherein said psychiatric disorder is major depressive disorder.

6. A method of diagnosing clinical depression in a subject, comprising:
determining activity of at least a portion of the anterior cingulate cortex of the subject;
determining an ability of the subject to respond to negative feedback and/or adjust behavior immediately after committing an error; and
diagnosing the subject as having clinical depression based on both the determination of the activity of the anterior cingulate cortex of the subject and the determination of the ability of the subject to respond to negative feedback and/or adjust behavior immediately after committing an error.

7. The method of claim 6, wherein the act of determining activity of at least a portion of the anterior cingulate cortex comprises determining activity of the rostral anterior cingulate cortex.

8. The method of claim 6, wherein the act of determining activity of at least a portion of the anterior cingulate cortex comprises using electroencephalography to determine activity.

9. The method of claim 8, wherein the act of determining activity of at least a portion of the anterior cingulate cortex comprises using quantitative electroencephalography to determine activity.

10. (canceled)

11. The method of claim 6, wherein the act of determining activity of at least a portion of the anterior cingulate cortex comprises using electromagnetic tomography to determine activity.

12. The method of claim 11, wherein the act of determining activity of at least a portion of the anterior cingulate cortex comprises using low-resolution electromagnetic tomography to determine activity.

13-16. (canceled)

17. The method of claim 6, wherein the act of determining an ability of the subject to respond to negative feedback and/or adjust behavior after committing errors comprises administering an Eriksen Flanker Task to the subject.

18. The method of claim 6, wherein the act of determining an ability of the subject to respond to negative feedback and/or adjust behavior after committing errors comprises administering a Stroop Task to the subject.

19. The method of claim 6, wherein the act of determining an ability of the subject to respond to negative feedback and/or adjust behavior after committing errors comprises administering a Counting Stroop Task to the subject.

20. The method of claim 6, wherein the act of determining an ability of the subject to respond to negative feedback and/or adjust behavior after committing errors comprises administering an Emotional Counting Stroop Task to the subject.

21. The method of claim 6, wherein the act of determining an ability of the subject to respond to negative feedback and/or adjust behavior after committing errors comprises administering an A-X Continuous Performance Test to the subject.

22. The method of claim 6, wherein the act of determining an ability of the subject to respond to negative feedback and/or adjust behavior after committing errors comprises administering a Go/NoGo Task to the subject.

23. The method of claim 6, wherein the act of diagnosing the subject comprises:
comparing the activity of at least a portion of the anterior cingulate cortex to a control value;
comparing the ability of the subject to respond to negative feedback and/or adjust behavior after committing errors to a control ability; and
identifying the subject as having clinical depression if both the activity of the at least a portion of the anterior cingulate cortex, and the ability of the subject to respond to negative feedback and/or adjust behavior after committing errors, each exceed their respective controls.

24. The method of claim 6, wherein the act of diagnosing the subject comprises:
calculating the difference between the activity of at least a portion of the anterior cingulate cortex of the subject and a control value;
calculating the difference between the ability of the subject to respond to negative feedback and/or adjust behavior after committing errors to a control ability;
combining the calculations to form a combined score; and
determining, using the combined score, whether the subject has clinical depression.

25. The method of claim 24, wherein the act of combining the calculations comprises equally weighting the activity of the at least a portion of the anterior cingulate cortex, and the ability of the subject to respond to negative feedback and/or adjust behavior after committing errors.

26. The method of claim 24, wherein the acts of calculating the difference between the activity of at least a portion of the anterior cingulate cortex of the subject and a control value, calculating the difference between the ability of the subject to respond to negative feedback and/or adjust behavior after committing errors to a control ability, and combining the calculations to form a combined score are performed using a computer.

27-32. (canceled)

33. A method of diagnosing clinical depression in a subject, comprising:
determining activity of the anterior cingulate cortex of the subject;
determining an ability of the subject to respond to negative feedback and/or adjust behavior after committing errors;
entering the determinations into a computer; and
receiving, from the computer, a probability assessment that the subject has clinical depression.

34. The method of claim 33, wherein the act of determining activity of at least a portion of the anterior cingulate cortex comprises determining activity of the rostral anterior cingulate cortex.

35. The method of claim 33, wherein the act of determining activity of at least a portion of the anterior cingulate cortex comprises using electroencephalography to determine activity.

36. The method of claim 35, wherein the act of determining activity of at least a portion of the anterior cingulate cortex comprises using low-resolution electromagnetic tomography to determine activity.

37. The method of claim 33, wherein the act of determining an ability of the subject to respond to negative feedback and/or adjust behavior after committing errors comprises administering an Eriksen Flanker Task to the subject.

38. The method of claim 33, wherein the act of determining an ability of the subject to respond to negative feedback and/or adjust behavior after committing errors comprises administering a Stroop Task to the subject.

39. (canceled)

40. A method, comprising:
receiving a determination of activity of the anterior cingulate cortex of a subject;
receiving a determination of the subject to respond to negative feedback and/or adjust behavior after committing errors; and
combining the determinations into a combined score.

41. The method of claim 40, wherein the act of receiving a determination of activity of at least a portion of the anterior cingulate cortex comprises receiving a determination of activity of the rostral anterior cingulate cortex.

42. The method of claim 40, wherein the act of receiving a determination of activity of at least a portion of the anterior cingulate cortex comprises receiving a determination of activity using electroencephalography to determine activity.

43. The method of claim 42, wherein the act of receiving a determination of activity of at least a portion of the anterior cingulate cortex comprises receiving a determination of activity using low-resolution electromagnetic tomography to determine activity.

44. The method of claim 40, wherein the act of receiving a determination of an ability of the subject to respond to negative feedback and/or adjust behavior after committing errors comprises receiving a determination of activity resulting from administering an Eriksen Flanker Task to the subject.

45. The method of claim 40, wherein the act of receiving a determination of an ability of the subject to respond to negative feedback and/or adjust behavior after committing errors comprises receiving a determination of activity resulting from administering a Stroop Task to the subject.

46. (canceled)

47. An article, comprising:
a computer-readable medium having a program stored thereon, which program comprises instructions for, when executed, causing a computer-driven system to perform acts of:
receiving a determination of activity of the anterior cingulate cortex of a subject;
receiving a determination of the subject to respond to negative feedback and/or adjust behavior after committing errors;
combining the determinations into a combined score; and
reporting the combined score.

48. An article, comprising:
a computer-readable medium having a program stored thereon, which program comprises instructions for, when executed, causing a computer-driven system to perform acts of:
determining activity of the anterior cingulate cortex of a subject;
determining ability of the subject to respond to negative feedback and/or adjust behavior after committing errors; and
identifying the subject as having clinical depression based on both the determination of the activity of the anterior cingulate cortex of the subject and the determination of the ability of the subject to respond to negative feedback and/or adjust behavior after committing errors.

49. A method, comprising:
determining activity of a portion of the brain of a subject;
determining an ability of the subject to respond to negative feedback and/or adjust behavior after committing errors; and
diagnosing the subject as having clinical depression based on both the determination of the activity of the portion of the brain of the subject and the determination of the ability of the subject to respond to negative feedback and/or adjust behavior after committing errors.

50. A method, comprising:
determining activity of a portion the brain of a subject using tomography;
determining an ability of the subject to respond to negative feedback and/or adjust behavior after committing errors; and
diagnosing the subject as having clinical depression based on both the determination of the activity of the brain of the subject and the determination of the ability of the subject to respond to negative feedback and/or adjust behavior after committing errors.
